(19) United States
(12) Patent Application Publication
(10) Pub. No.: US 2015/0173406 A1
(43) Pub. Date: Jun. 25, 2015

(54) HYPOALLERGENIC FOOD-GRADE PROTEIN MATRICES AND USES THEREOF

(71) Applicants: North Carolina State University, Raleigh, NC (US); The University of North Carolina at Chapel Hill, Chapel Hill, NC (US); The United States of America, as represented by the Secretary of Agriculture, Washigton, DC (US)

(72) Inventors: Mary Ann Lila, Raleigh, NC (US); Mary H. Grace, Raleigh, NC (US); Jack P. Davis, Raleigh, NC (US); Wesley Burks, Chapel Hill, NC (US); Michael D. Kulis, Chapel Hill, NC (US)

(73) Assignees: North Carolina State University, Raleigh, NC (US); The University of North Carolina at Chapel Hill, Chapel HIll, NC (US); The United States of America, as represented by the Secretary of Agriculture, Washington, DC (US)

(21) Appl. No.: 14/413,859

(22) PCT Filed: Jul. 9, 2013
(86) PCT No.: PCT/US2013/049802

§ 371 Date: Jan. 9, 2015

Related U.S. Application Data
(60) Provisional application No. 61/669,353, filed on Jul. 9, 2012.

(51) Int. Cl. A23L 1/30; A61K 36/45; A23L 1/29; A23L 1/36; A23L 1/20; A61K 39/35; A61K 36/185

(52) U.S. Cl. CPC A23L 1/3002; A61K 39/35; A61K 36/45; A61K 36/185; A23L 1/366; A23L 1/20; A23L 1/296; A23V 2002/00

(57) ABSTRACT

This invention relates generally to the discovery of improved food-grade protein matrices with reduced allergeiiicity and uses thereof including immunotherapy.

### CROSS REFERENCE TO RELATED APPLICATIONS

This application claims the benefit of U.S. Provisional Appn. 61/669,353 filed Jul. 9, 2012, Lila et al., entitled “Hypoallergenic Food-Grade Protein Matrices and Uses Thereof” having Atty. Docket No. NS12004USV, which is hereby incorporated by reference in its entirety.

### 1. FIELD OF THE INVENTION

This invention relates generally to the discovery of improved food-grade protein matrices with reduced allergenicity and uses thereof including immunotherapy.

### 2. BACKGROUND OF THE INVENTION

### 2.1. Introduction

Peanut allergies. Peanut (Arachis hypogeae L.) allergy is estimated to affect roughly 1% of children in North America and the UK. Sicherer, S. H.: Sampson, H. A., Peanut allergy: Emerging concepts and approaches for an apparent epidemic. J. Allergy Clin. Immunol. 2007, 120 (3), 491-503. Peanut is considered one of the most severe food allergies, with the majority of fatal food allergic reactions reported in the US attributable to peanuts. Bock, S. A.; Munoz-Furlong, A.; Sampson, H. A., Further fatalities caused by anaphylactic reactions to food, 2001-2006. J. Allergy Clin. Immunol. 2007, 119 (4), 1016-1018. Furthermore, unlike most food allergies, only about 20% of children allergic to peanuts outgrow this disorder. Skolnick, H. S.; Conover-Walker, M. K.; Koerner, C. B.; Sampson. H. A.; Burks, W.; Wood, R. A., The natural history of peanut allergy. J. Allergy Clin. Immunol. 2001, 107 (2), 367-374. Peanut allergic reactions involve an immunoglobulin E (IgE)-mediated immunological response to various proteins within the edible seed. To date, 11 allergenic peanut proteins have been characterized and cloned and are designated as Ara h 1-11. Peanut seed are typically 24-29% protein by weight, of which 80-90% these proteins are the allergenic storage proteins, i.e. Ara h 1-4. Ara h 1 and h 2 are considered major allergens as 70-90% of allergic patient's sera respond with positive IgE binding. Koppelman, S. J.; Vlooswijk, R. A. A.: Knippels, L. M. J.; Hessing. M.; Knol, E. F.: van Reijsen, F. C.; Bruijnzeel-Koomen, C., Quantification of major peanut allergens Ara h 1 and Ara h 2 in the peanut varieties Runner, Spanish, Virginia, and Valencia, bred in different parts of the world. Allergy 2001, 56 (2), 132-137; Maleki, S. J.; Kopper, R. A.; Shin, D. S.; Park, C. W.; Compadre, C. M.; Sampson, H.; Burks, A. W.; Bannon, G. A., Structure of the major peanut allergen Ara h 1 may protect IgE-binding epitopes from degradation. J. Immunol. 2000, 164 (11), 5844-5849.

A primary mechanism of allergenic food proteins is IgE binding coupled with cross-linking on the surface of mast cells that ultimately results in downstream cascades responsible for the allergenic response. Burks, A. W.; Laubach, S.; Jones, S. M., Oral tolerance, food allergy, and immunotherapy: Implications for future treatment. J. Allergy Clin. Immunol. 2008, 121 (6), 1344-1350; Sicherer, S. H.; Sampson. H. A., Food allergy. J. Allergy Clin. Immunol. 2010, 125 (2), S116-S125.

Protein segments that bind IgE are referred to as epitopes. Epitopes are attributed to either a given linear sequence of amino acids within the protein, or to a portion of the three dimensional structure of the protein, and are designated as either linear or conformational, respectively. Factors influencing protein allergenicity include the capacity of a protein to bind IgE, stimulate production of IgE and resist digestion within the gastrointestinal tract. Sicherer 2010; Sathe, S. K.; Sharma, G. M., Effects of food processing on food allergens. Mol. Nutr. Food Res. 2009, 53 (8), 970-978.

Strategies to Reduce or Treat Peanut Allergies in Affected Patients.

Because peanut allergies can present life-threatening consequences, there is intense interest in developing therapeutic strategies that could reduce the danger and severity of the allergic reaction to peanuts in sensitive patients. Various processing-based strategies are being investigated for the potential to modify/improve the allergenic profiles of proteins. Sathe 2009; Mills, E. N. C.; Mackie, A. R., The impact of processing on allergenicity of food. Curr. Opin. Allergy Clin. Immunol. 2008, 8 (3). 249-253. Some examples include heat induced aggregation, enzymatic hydrolysis and controlled Maillard type modifications. Lemon-Mule, H.; Sampson, H. A.; Sicherer, S. H.; Shreffler, W. G.; Noone, S.; Nowak-Wegrzyn, A., Immunologic changes in children with egg allergy ingesting extensively heated egg. J. Allergy Clin. Immunol. 2008, 122 (5), 977-983: Mouecoucou, J.; Fremont, S.; Sanchez, C.; Villaume, C.: Mejean. L., In vitro allergenicity of peanut after hydrolysis in the presence of polysaccharides. Clin. Exp. Allergy 2004, 34 (9). 1429-1437; Taheri-Kafrani, A.; Gaudin, J. C.; Rabesona. H.; Nioi, C.; Agarwal. D.; Drouet, M.: Chobert. J. M.: Bordbar, A. K.; Haertlft, T., Effects of Heating and Glycation of beta-Lactoglobulin on Its Recognition by IgE of Sera from Cow Milk Allergy Patients. J. Agric. Food Chem. 2009, 57 (11), 4974-4982.

Oral immunotherapy (OIT), which involves highly regulated administration, in a clinical setting, of very small doses of allergenic proteins, including peanut protein in the form of peanut flour, is a strategy that has recently shown promise for desensitizing some allergic patients, so as to attenuate a potentially life threatening anaphylactic reaction to a chance ingestion of allergenic foods including peanut products. Varshney, P.; Jones, S. M.; Scurlock, A. M.; Perry. T. T.; Kemper, A.: Steele, P.: Hiegel. A.; Kamilaris, J.: Carlisle, S.; Yue. X. H.: Kulis, M.; Pons. L.; Vickery, B.; Burks, A. W., A randomized controlled study of peanut oral immunotherapy: Clinical desensitization and modulation of the allergic response. J. Allergy Clin. Immunol. 2011, 127 (3), 654-660. However, OIT carries significant risks of side effects, including gastrointestinal problems, wheezing, and even anaphylactic shock; these barriers preclude rapid dissemination of the technology beyond highly controlled clinical settings.

The utility of polyphenol-protein interactions have been exploited by humans since the Greeks in 4th century BC used oak galls for tanning leather hides. Douat-Casassus. C., Chassaing, S., Di Primo, C. & Quideau, S. 2009. Specific or nonspecific protein polyphenol interactions? Discrimination in real time by surface plasmon resonance. ChemBioChem 10: 2321-2324: Van-Driel Murray, C. 2000. Leatherwork and skin products. In: Ancient Egyptian Materials and Technology (eds. PT Nicholson and I Shaw). Pp. 299-319. Cambridge University Press, Cambridge, UK. However, a practical means for efficient modification of allergenic protein epitopes with natural plant-accumulated phytochemicals has not been accomplished previously.

### 3. SUMMARY OF THE INVENTION

In particular non-limiting embodiments, the present invention provides a solid foodstuff with reduced allergenicity which comprises a solid protein rich ingredient treated with fruit/vegetable polyphenolic phytochemicals.

The invention also provides a method for reducing the allergenicity of a solid protein rich foodstuff which comprises treating the solid protein rich foodstuff with fruit/vegetable polyphenolic phytochemicals. The solid protein rich foodstuff may be treated by mixing with a juice or extract rich in fruit/vegetable polyphenolic phytochemicals to form a complex between the solid protein rich foodstuff and the fruit/vegetable polyphenolic phytochemicals which may include centrifugation and lyophilization (or freeze drying) of the complex between the solid protein rich foodstuff and the fruit/vegetable polyphenolic phytochemicals.

In another non-limiting embodiment, the invention provides method of preparing an infant formula or baby food with reduced allergenicity which comprises treating a solid protein rich foodstuff with fruit/vegetable polyphenolic phytochemicals so as to form a complex between the solid protein rich foodstuff and the fruit/vegetable polyphenolic phytochemicals; and using the complex to prepare the infant formula or baby food with reduced allergenicity.

The invention also provides a method of reducing an allergic reaction to a protein rich foodstuff in a subject which comprises (a) treating the solid protein rich foodstuff with fruit/vegetable polyphenolic phytochemicals to form a complex between the solid protein rich foodstuff and the fruit/vegetable polyphenolic phytochemicals; and (b) administering the complex to the subject so as to build a tolerance to the protein rich foodstuff thereby reducing the allergic reaction to the protein rich foodstuff.

In the foodstuffs and methods above, the solid protein rich ingredient may be a legume or nut protein, such as a peanut protein, a peanut flour, or a tree nut protein. The solid protein rich ingredient may be a soy protein, an egg protein, a milk protein, a wheat protein, a fish protein or a shellfish protein. The uses of such foods may be any high protein foods such as protein bars or protein drinks including infant formula.

Non-limiting examples of solid protein rich ingredients include defatted soy flour, hemp protein, oat bran, peanut protein, peanut flour (PNF), pea protein isolate (PPI), rice protein concentrate (RPC), or soy protein isolate (SPI). The fruit/vegetable polyphenolic phytochemicals may be an A-type proanthocyanidin source such as: cranberry juice, cinnamon extract; a B-type proanthocyanidin source such as: blueberry or chokeberry (Aronia), grape seed, green tea (catechins); an anthocyanin source such as: black currant or strawberry; or a smaller molecular weight (MW) source of polyphenolics and carotenoids such as mango. In one non-limiting embodiment, it may be an extract from a berry extract such as a cranberry extract or black currant extract. Examples of fruit/vegetable polyphenolic phytochemical complexes include, but are not limited to, apple—SPI; black currant—SPI; blueberry—pea; blueberry—SPI; chokeberry—SPI; cinnamon—SPI; cranberry-oat bran; cranberry—PPI; cranberry—SPI; cranberry-whey: cranberry-wheat bran; cranberry-wheat germ defatted soy flour; grape—PPI; grape—SPI; green tea—PPI; guava—SPI; kiwi—SPI; lemon—SPI; maqui berry—PPI; passion fruit—SPI; pear—SPI; pomegranate—SPI; rhubarb—SPI; and strawberry—SPI.

### 4. BRIEF DESCRIPTION OF THE FIGURES

FIG. 1: Three peanut flours before (top row) and after (bottom row) complexing with the polyphenols present in black currant juice concentrate. 25× dilution, 30 g flour/L, and only 15 minutes of processing time.

FIG. 2: Effect of peanut flour concentration on the anthocyanins sorbed from a black currant juice concentrate. Two dilutions of the commercial juice concentrate (100× and 150×) were tested at two flour concentrations (50 or 30 g peanut flour per L).

FIG. 3: Effect of black currant juice concentration on sorbing capacity of peanut flour (30 g flour/L). Both medium and dark roasted flours demonstrated higher sorption capacity than light roasted flours. The most concentrated fruit extract (25×) resulted in the highest concentration of anthocyanins in the treated matrix.

FIG. 4: Binding capacity of peanut flour as compared to defatted soy flour. Peanut flours demonstrate comparable or higher capacity to sorb anthocyanins from natural sources.

FIG. 5: Western blot of polyphenolic modified peanut flour matrix preparations. Light roast peanut flour prepared with black currant or cranberry concentrates. See text for details.

FIG. 6: FTIR graphs demonstrating alterations of peanut epitopes after complexing with proanthocyanidin-rich cranberry extracts (6A) or with primarily anthocyanin-rich black currant extracts (6B).

FIG. 7: Basophil degranulation assay using whole blood from peanut allergic children. 1 mg of modified flour complexed with polyphenols was added to 200 μL basophil media, then added to the whole blood. Degranulation was monitored by flow cytometry. The cranberry polyphenol-enriched peanut flours (at 2× and 5× dilutions of cranberry peanut flour) have significantly reduced (p-value <0.05) basophil activation capacity, demonstrating an approximately ˜50% decrease in median as compared to the unmodified peanut flour.

FIG. 8: Basophil degranulation assay comparing modified peanut flours produced with cranberry anthocyanin or proanthocyanidin fractions, with or without tyrosinase enzyme treatment. Both peanut-allergic patients show a substantial decrease in basophil degranulation when the anthocyanin/proanthocyanidins molecules are complexed to the flour in the presence of tyrosinase enzyme. These findings indicate that further modifications to the peanut proteins can further improve efficacy.

FIG. 9: MMCP-1 levels in serum 45 minutes following oral challenge with non-modified peanut flour (PNF) or peanut protein-phytoactive aggregates (PPPA) in peanut-allergic mice.

FIG. 10: Th2- and Th1-type cytokines secreted from peanut-allergic mouse splenocytes with stimulation from peanut (PN) or PN modified cranberry juice (CB).

### 5. DETAILED DESCRIPTION OF THE INVENTION

One impetus for our novel strategy (described below) is that the deliberately engineered complexation of proteins (e.g. peanut epitopes) with flavonoids and other natural polyphenolic molecules derived from fruits or vegetable sources, can effectively mask and modify the epitopes in peanut flours to further condition the degree of allergenicity in a highly controlled way. These generally recognized as safe (GRAS) polyphenolic compounds, when complexed to peanut proteins in a prepared preroasted flour-type matrix, block access of IgE to epitopes, modify protein three dimensional folds, modify solubility, modify protein digestion patterns, etc. all of which could influence allergenic potential. Previously, limited work with peanut protein suggests that reactions with model phenolic compounds such as caffeic, chlorogenic and ferulic acids can result in insoluble protein complexes leading to decreased IgE binding in soluble fractions. Chung, S. Y.; Champagne, E. T., Reducing the allergenic capacity of peanut extracts and liquid peanut butter by phenolic compounds. Food Chem. 2009, 115 (4), 1345-1349.

Innovation

Many of the allergenic epitopes of peanut (as present in roasted peanut flours) may be blocked and modified, by complexing them with the health-protective natural polyphenolic phytochemicals from fruit or vegetable extracts. As a result, such fruit/vegetable-enriched peanut flours have the potential to, gradually and in a rigorously-controlled, stepwise progression of allergenicity levels. “prime” the immune system and provide the benefit of tolerance with less risk of inducing an extreme allergenic reaction to the protein. In addition, the treatment will involve simple oral intake of food-grade ingredients, and will not require intrusive procedures for the patient. The covalent binding of polyphenols to the proteins of peanut (or other potential allergen) may effectively mask and protect reactive epitopes from disassociation during metabolism after ingestion by an allergic individual. The technique captures, concentrates in a shelf-stable format, and preserves the biologically-active natural flavonoids in fruits/vegetables (examples—anthocyanins. proanthocyanidins, sesquiterpene lactones, gingerols, ellagic and other phenolic acids) by binding them to peanut proteins in a defined preroasted flour matrix. Our simple process uses mixing, centrifugation and lyophilization to complex the defined peanut flours with fruit/plant juiced extracts. The engineered stoichiometry of this process achieves a step-wise gradient of different peanut flour preparations with increasing levels of complexation, and therefore, gradually decreasing levels of allergenicity.

5.1. Compositions

The invention provides compositions for the present invention provides a solid foodstuff with reduced allergenicity which comprises a solid protein rich ingredient treated with fruit/vegetable polyphenolic phytochemicals.

5.2. Methods

The invention also provides a method for reducing the allergenicity of a solid protein rich foodstuff which comprises treating the solid protein rich foodstuff with fruit/vegetable polyphenolic phytochemicals. The solid protein rich foodstuff may be treated by mixing with a juice or extract rich in fruit/vegetable polyphenolic phytochemicals to form a complex between the solid protein rich foodstuff and the fruit/vegetable polyphenolic phytochemicals which may include centrifugation and lyophilization (or freeze drying) of the complex between the solid protein rich foodstuff and the fruit/vegetable polyphenolic phytochemicals. Alternative methods of drying such as forced air drying, low heat air drying, or low heat oven drying are suitable also.

The invention also provides a method of reducing an allergic reaction to a protein rich foodstuff in a subject which comprises (a) treating the solid protein rich foodstuff with fruit/vegetable polyphenolic phytochemicals to form a complex between the solid protein rich foodstuff and the fruit/vegetable polyphenolic phytochemicals; and (b) administering the complex to the subject so as to build a tolerance to the protein rich foodstuff thereby reducing the allergic reaction to the protein rich foodstuff.

The article “a” and “an” are used herein to refer to one or more than one (i.e., to at least one) of the grammatical object of the article. By way of example, “an element” means one or more element.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. The present invention may suitably comprise, consist of, or consist essentially of, the steps and/or reagents described in the claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The following Examples further illustrate the invention and are not intended to limit the scope of the invention.

### 6. EXAMPLES

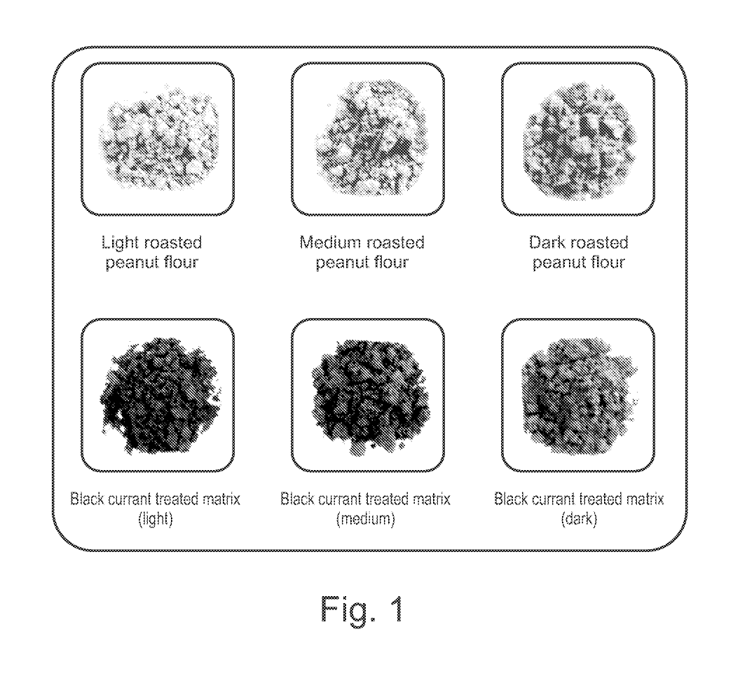

These polyphenolic phytochemicals bind stably to the peanut proteins via a combination of interactions involving hydrophobic and hydrogen bonding as well as covalent interactions, while all water and sugars in the original juiced fruits or vegetable extracts pass through and are excluded from the final polyphenol-enriched peanut flour matrix. The polyphenolic-protein interaction changes the physicochemical and structural properties of proteins. The enhanced peanut matrix is lyophilized to provide a shelf-stable, low-caloric and food grade ingredient (FIG. 1). Bioactivity and integrity of the phytochemical components, which would be quickly degraded in a fresh fruit or vegetable juice product, are preserved long term in the functional matrix.

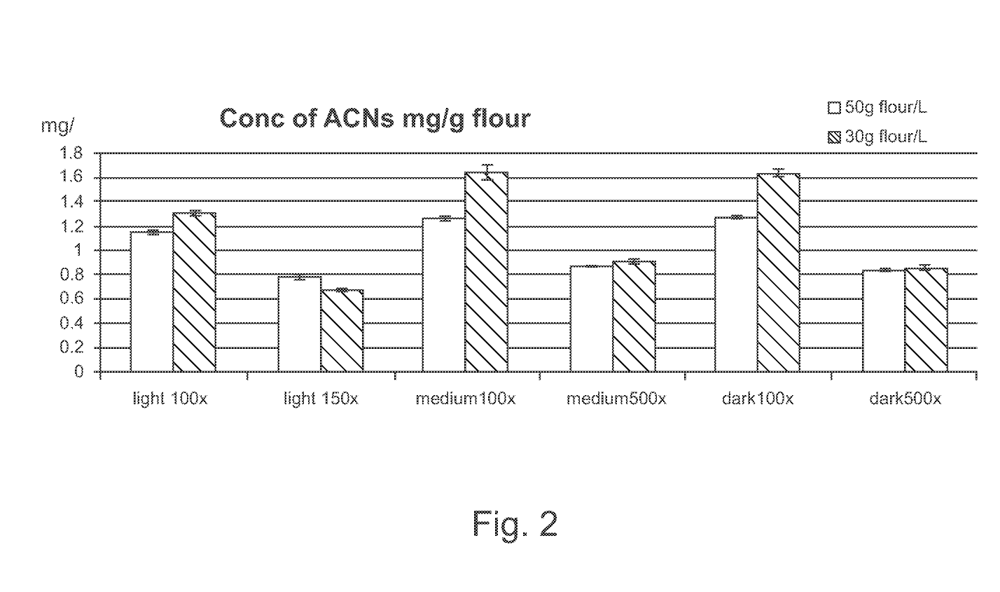

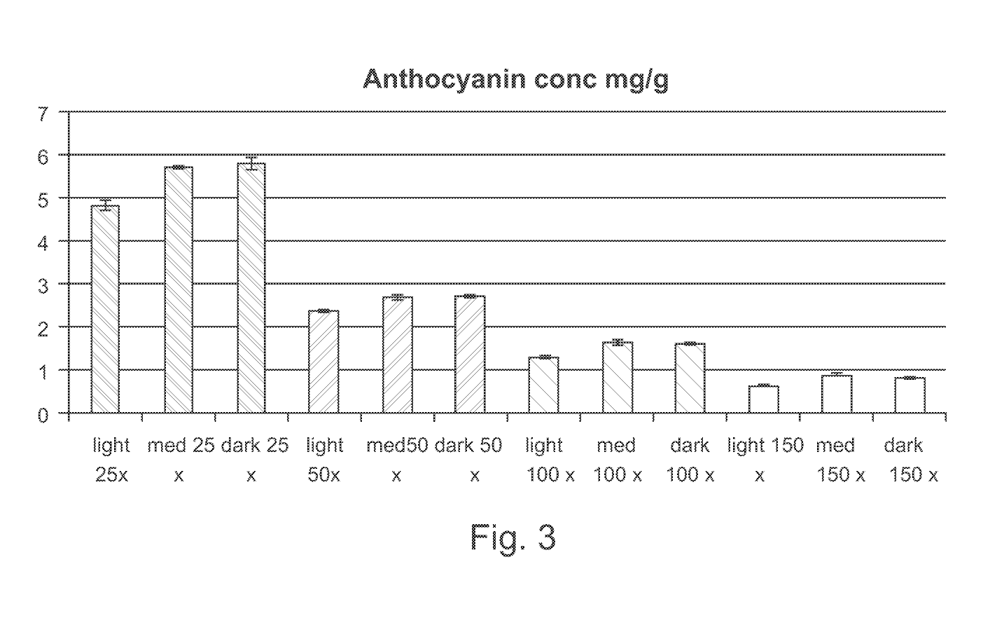

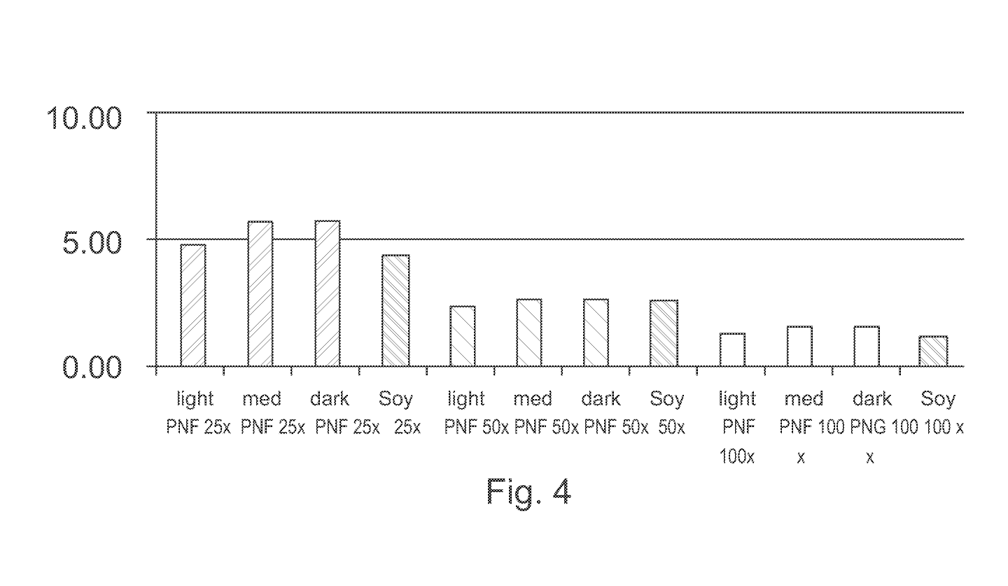

To date, fruit concentrates from black currant, cranberry, and other anthocyanin and proanthocyanidin-rich natural sources, have been complexed with various grades of peanut flour to determine maximum sorption and protein binding (FIGS. 2-4). Peanut flours with different roast colors, i.e. light medium or dark, demonstrate enough variability in sorption of anthocyanins (ANC) to strongly suggest roast color is an important variable when considering polyphenolic adsorption experiments. Medium and dark roasted peanut flours seem to demonstrate a higher capacity to sorb ANC from natural fruit sources, but more work is needed to confirm these findings. Peanut flours are commercially available, high protein ingredients prepared from dry roasted peanuts that have been partially defatted, and the hydrophilic and lipophilic antioxidant properties of these materials have recently been documented. Davis, J. P.; Dean, L. L.; Price, K. M.; Sanders, T. H., Roast effects on the hydrophilic and lipophilic antioxidant capacities of peanut flours, blanched peanut seed and peanut skins. Food Chem. 2010, 119 (2), 539-547. Our experiments have demonstrated that peanut flours are excellent substrates for stably binding fruit flavonoids. Over 1.6 mg/anthocyanins are captured per g of peanut flour, which effectively stabilizes the biologically-active pigments from 1-2 servings of fruit in just a few grams of matrix. This pre-roasted peanut flour matrix with stably-bound fruit flavonoids complexed with the proteins could be administered in clinical settings to patients to reliably control the exposure to peanut allergens (in a food grade ingredient, not a pharmaceutical preparation), to allow gradual stepwise desensitization.

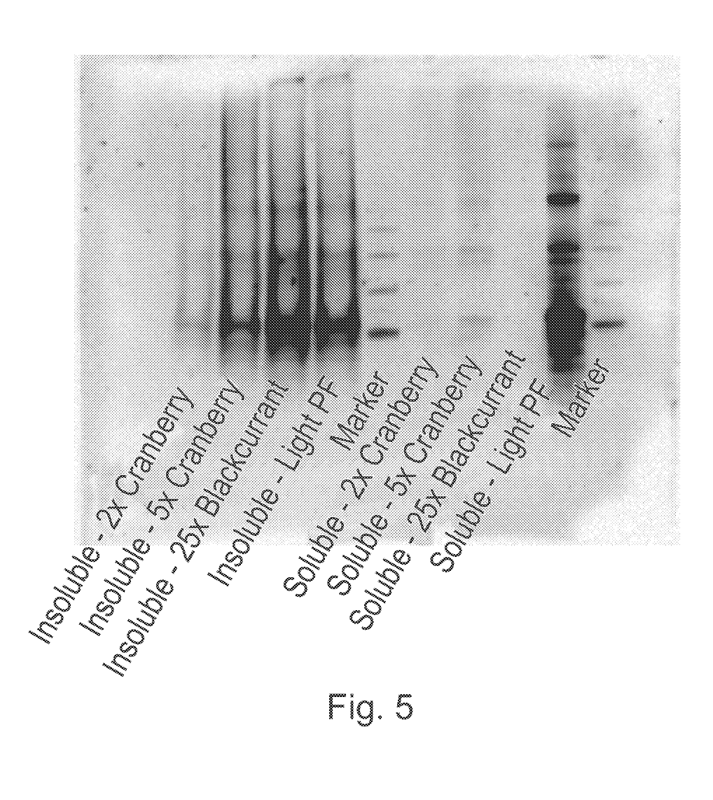

Additional assays including Western Blots (FIG. 5) indicate that the stable polyphenolic binding to epitopes in the enhanced matrix indeed decreases IgE binding.

Samples Analyzed:
Light Roast, 12% Oil, Peanut Flour (unmodified control)
Light Roast, 12% Oil, Peanut Flour modified via polyphenolic sorption process
25× Black currant/Peanut Flour
5× Cranberry/Peanut Flour
2× Cranberry/Peanut Flour

Sample Preparation:

Flours (control and modified) were prepared into 1% dispersions by adding 0.1 g flour to 10 mL dH₂O. Dispersions were extensively vortexed and then centrifuged at 1075 rpm for 15 min. The supernatants were poured off and designated as soluble fractions. Insoluble pellets were also collected for subsequent testing. The insoluble pellets were dried at 130° C. for 2 hr. Dried insoluble pellets were rehydrated to approximate 1% solutions with SDS PAGE buffer (0.05 g pellet+5 mL SDS-PAGE Buffer). Solutions were extensively vortexed to ensure the dried pellets were soluble in the SDS PAGE buffer and these re-solubilized dried pellets were designated as insoluble fractions.

Sodium Dodecyl Sulfate-Polyacrylamide Gel Electrophoresis (SDS-PAGE).

Soluble fractions and insoluble fractions were diluted using NuPAGE® LDS Sample Buffer (4×) and NuPAGE® Sample reducing Agent (10×) according to the NuPAGE® Technical Guide. All samples were incubated at 70° C./10 min prior to SDS PAGE. For all soluble fractions, an estimated target protein concentration of 5 μg (based on unmodified control and determined by BCA Protein Assay) was loaded per well in NuPAGE® 1.0 mm×10 well 4-12% Bis-Tris Gels (Invitrogen, Carlsbad, Calif.). BCA protein data for soluble fractions prepared from the modified flours was not reliable due to colorimetric interference. As such, a volume equal to that loaded for the unmodified control, was loaded for each of the soluble fractions prepared from modified flours. To better understand actual protein contents/well, samples were subsequently measured for soluble nitrogen using a 2400 CHN Elemental Analyzer (Perkin Elmer, Norwalk, Conn.). Data was converted to soluble protein using the accepted nitrogen/protein conversion factor of 5.43 for peanut proteins. From this analysis, final protein concentrations of 2.75, 0.46, 0.46 and 0.46 μg/well for light peanut flour (unmodified control), 2× cranberry, 5× cranberry and 25× black currant were determined to have been loaded. For insoluble fractions, equal volumes of the rehydrated dried pellets were analyzed, and the final protein concentration/well were estimated to be 679.31, 678.24, 643.10 and 739.06 μg protein/well for light peanut flour (unmodified control). 2× cranberry, 5× cranberry and 25× black currant as determined by using a 2400 CHN Elemental Analyzer and a nitrogen/protein conversion factor of 5.43.

Final Volumes Analyzed:

Soluble fractions were diluted accordingly prior to SDS-PAGE analyses:
65 μL of soluble fraction
25 μL of SDS PAGE buffer
10 μL of DTT (reducing agent)
20 μL of the above solution was then loaded per lane.

Insoluble fractions were prepared accordingly prior to SDS-PAGE analyses:
65 μL of insoluble fraction rehydrated in SDS PAGE Buffer
25 μL of additional SDS PAGE buffer
10 μL of DTT (reducing agent)
20 μl of the above solution was then loaded per lane.

Electrophoresis conditions were 200 V for 35 min in MES SDS Running Buffer (1×). MagicMarke™XP Western Standard was used as a molecular weight marker.

Western Blotting.

Following SDS PAGE, gels were transferred onto an Immobilon® Transfer Membrane at 25 V for 90 min using an XCell II™ Blot Module. Ponceau S Solution was used to stain the gel for 5 min. The membranes were placed in the diluted human sera (pool of confirmed peanut allergic patients) overnight and then incubated in diluted Biotin-Labeled affinity purified antibody to human IgE. Samples were then incubated in diluted NeutrAvidin™ Horseradish Peroxidase Conjugate for 30 min, and blots were then submerged in SuperSignal® West Pico Chemiluminescent Substrate for 3 min. A Chemi Doc® Imaging System was used for image capture.

Discussion.

The Western Blot protocol used in this experiment ultimately requires a protein to be soluble prior to analysis; however, much of peanut flour is not soluble in typical aqueous extracts. Since it is important to understand the allergenic potential of peanut flour in totality, not just the soluble portion, the protocol described above was designed to try and best analyze peanut flour in totality. Soluble and insoluble fractions were first analyzed via SDS-PAGE, which separates proteins according to their molecular weight. Protein content of the soluble fraction for the unmodified control flour was determined by the BCA protein assay. This information was used to then load 5 μg of protein/lane for this control sample in the SDS PAGE assay. BCA protein data for soluble fractions prepared from the modified flours is not reliable due to colorimetric interference. As such, a volume equal to that loaded for the unmodified control, was loaded for each of the soluble fractions. Nitrogen content on soluble fractions is analyzed to determine amount of protein loaded onto gel for all flours. To better understand actual protein contents/well, samples were subsequently measured for soluble nitrogen using a 2400 CHN Elemental Analyzer (Perkin Elmer, Norwalk, Conn.). Data was converted to soluble protein using the accepted nitrogen/protein conversion factor of 5.43 for peanut proteins. From this analysis. Table 1 shows final protein concentrations of 2.75, 0.46, 0.46 and 0.46 μg/well for light peanut flour (unmodified control), 2× cranberry, 5× cranberry and 25× black currant were determined to have been loaded.

TABLE 1
Protein (μg) loaded per well for Western Blot.

| | | reconstituted insoluble |
|---|---|---|
| | 1% soluble fractions | fractions |
| Light Peanut Flour | 2.75 | 679.31 |
| 2x Cranberry | 0.46 | 678.24 |
| 5x Cranberry | 0.46 | 643.10 |
| 25x Black currant | 0.46 | 739.06 |

Following SDS-PAGE, the gel was then transferred onto a PVDF membrane and standard Western Blotting Protocol was followed as described above. Samples were exposed to a pool of blood sera derived from patients with confirmed peanut allergy. Increasing “darkness” on the blot indicates increased IgE binding. For soluble fractions, the unmodified flour bound much more IgE than any of the modified flours. For soluble fractions, both 2× cranberry and 25× black currant showed substantial reduction in IgE binding such that only trace IgE binding could be observed. For soluble cranberry samples, a greater reduction in IgE binding was observed for the 2× (less dilute) sample as compared to the 5× (more dilute) sample which suggests exposure to more concentrated phenolic compounds resulted in more intensely modified flours which bound less IgE. For insoluble fractions, the greatest reduction in IgE binding was observed for the 2× cranberry. Both control, 5× cranberry, and 25× black currant showed heavy IgE binding at ˜20 kDa the approximate molecular weight of the primary peanut allergen, Ara h 2.

In general, reduction in IgE binding is attributed to polyphenolic compounds complexing with the proteins and masking epitopes and/or modifying protein structure such that IgE binding potential is modified.

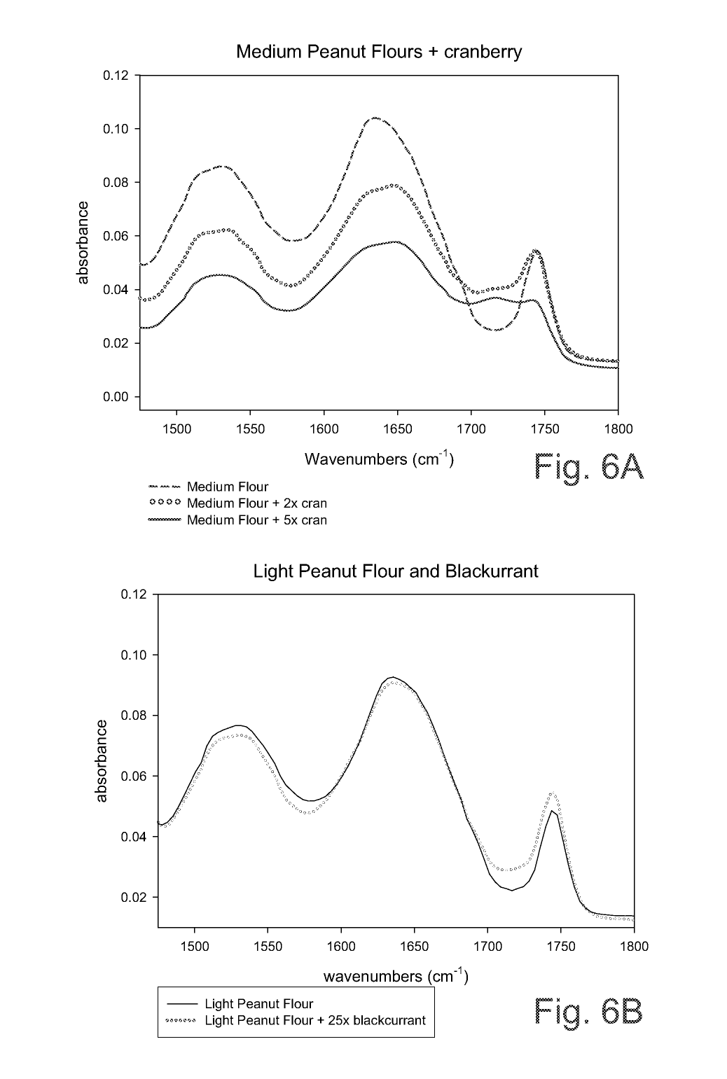

In addition, as evidenced in the FTIR spectra below (FIG. 6), peanut proteins are clearly altered after complexing with the proanthocyanidin-rich fruit extracts from cranberry, whereas complexing with anthocyanin-rich black currant extracts does not alter the spectra. Hypoallergenic foods have excellent potential to serve in therapeutic applications (van Putten, M. C.; Frewer, L. J.; Gilissen, L.; Gremmen, B.; Peijnenburg, A.; Wichers, H. J., Novel foods and food allergies: A review of the issues. Trends Food Sci. Technol. 2006, 17 (6), 289-299) and our invention establishes that complexing pre-roasted defined composition peanut flours with natural food grade phytochemicals such as those available in fruit/vegetable extracts has strong potential to reduce IgE binding potential. The potential for phenolic compounds, such as those naturally present in fruits and vegetables, to modify peanut proteins and therefore reduce their allergenic potential is logical based on known polyphenol-protein binding potential (the principle of tanning leather). However, the innovation described in this document presents a novel technology for simply, stably, and predictably generating a hypoallergenic peanut edible matrix for immunotherapy applications using cost-effective methods. Unlike other processes, this unique technology could be readily applied on a commercial scale to generate peanut flour materials specifically tailored for immunotherapy applications. Previous work has shown that peanut protein-polyphenolic complexes are less soluble, hence soluble extracts prepared from such complexes display reduced IgE binding capacity as compared to nonmodified soluble extracts (Chung, S. Y.; Champagne, E. T., Reducing the allergenic capacity of peanut extracts and liquid peanut butter by phenolic compounds. Food Chem. 2009, 115 (4). 1345-1349) however, recent basophil degranulation assays suggest “solid state” measurements are necessary to truly determine hypoallergenic potential. The novel procedure described here could be further modified to covalently link polyphenolics compounds to peanut protein allergens. Covalent complexes prepared with beta-lactoglobulin (primary whey protein) and sour cherry phenolics were recently shown to reduce basophil degranulation. Tantoush, Z., et al., Digestibility and allergenicity of beta-lactoglobulin following laccase-mediated cross-linking in the presence of sour cherry phenolics. Food Chemistry, 2011. 125(1): p. 84-91. Digestability, which is another important consideration in the allergenic potential of a protein, was also shown to be affected by phenolic complexation.

With a conservative estimate that 1% of children in the Western world have a peanut allergy (many studies put this number substantially higher), and with many indicators suggesting these number are increasing both in the Western world and in developing countries, the demand for modified, hypoallergenic peanut protein substrates could be significant. Furthermore, this approach could also be readily applied to other common food allergies including milk, egg, soy, etc. We have developed this novel peanut matrix as a therapeutic agent for peanut allergy treatment, and propose to further screen and validate the technology in collaborative research to fully establish the utility and potential of a food grade pre-roasted peanut flour matrix with stepwise defined levels of reduced allergenicity. Recent data compiled for our enriched, polyphenol-fortified food-grade peanut flour ingredient indicate that it may be useful for OIT by medical professionals to gradually desensitize the human immune system to build tolerance to peanut allergens, while minimizing adverse side effects currently experienced with non-modified peanut flours. In addition, the process creates a polyphenolic-peanut complex which can readily be used as an ingredient in applications where peanuts are already being used (nutritional bars, confections, etc.) with an added bonus of co-delivery of recognized bioactive phytochemicals from natural sources.

The enhanced peanut flour ingredient is highly shelf stable and maintains the bioactivity and integrity of the fruit constituents well over a year in storage. SDS-PAGE and Western Blot assays, modified to allow analysis of whole peanut flour (soluble and insoluble phases) were performed. The enhanced polyphenol-peanut ingredient, enriched with natural A-type proanthocyanidins from cranberry, demonstrated (after exposure to a pool of blood sera from peanut-allergic patients) substantial reduction in immunoglobulin E (IgE) binding, such that only trace IgE binding could be observed (FIG. 5). Reduction in IgE binding was correlated with the concentration of bound polyphenolics in the matrix, which masked epitopes and/or modified protein structure. Additionally, attenuated total reflectance Fourier transform infrared spectroscopy (ATR-FTIR) spectra suggested that the secondary structures of the proteins were significantly altered after complexation with proanthocyanidin-rich extracts, but not by extracts containing only anthocyanins and other flavonoids (FIG. 6).

Based on these promising outcomes, robust, state-of-the-art bioassays using basophils (antigen specific cells in IgE sensitized individuals) as highly relevant biomarkers of IgE-mediated hypersensitivity was conducted.

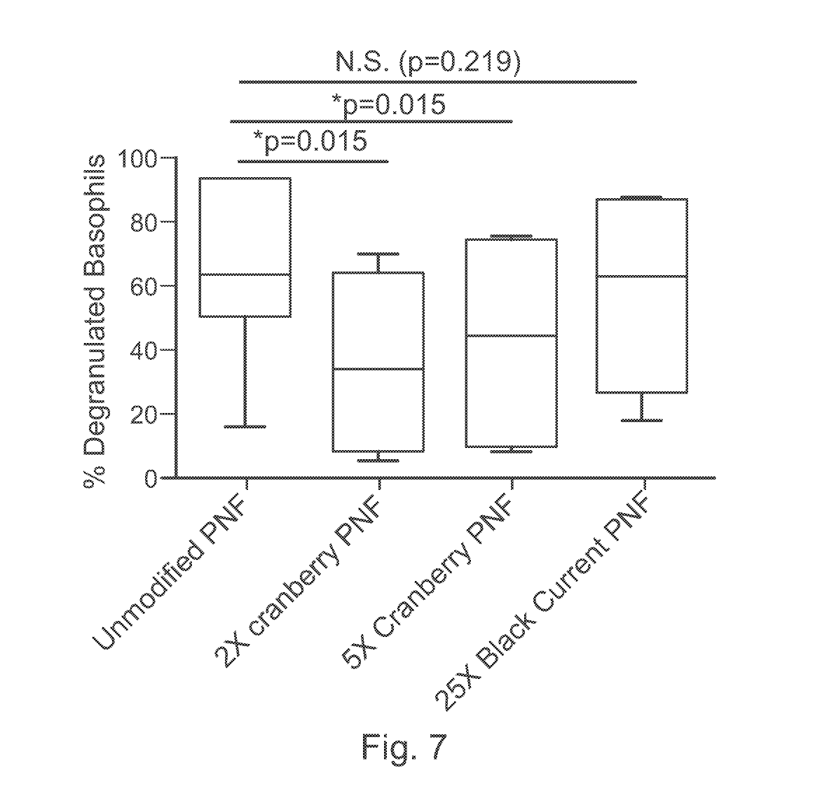

Specifically, the ability of the polyphenol-fortified peanut flours to attenuate degranulation and histamine release from basophils is gauged to determine efficacy. The assay was successfully adapted to allow testing of the modified peanut flours in the solid phase, since the peanut proteins in the modified flours are largely insoluble. Modified peanut flours developed using proanthocyanidin-rich cranberry juice concentrate (CJC) [at two dilutions, 2× and 5×] showed less degranulation, compared to treatments provoked with unmodified peanut flour preparations or peanut flours complexed with black currant polyphenolics after sampling blood from seven peanut allergic patients (FIG. 7).

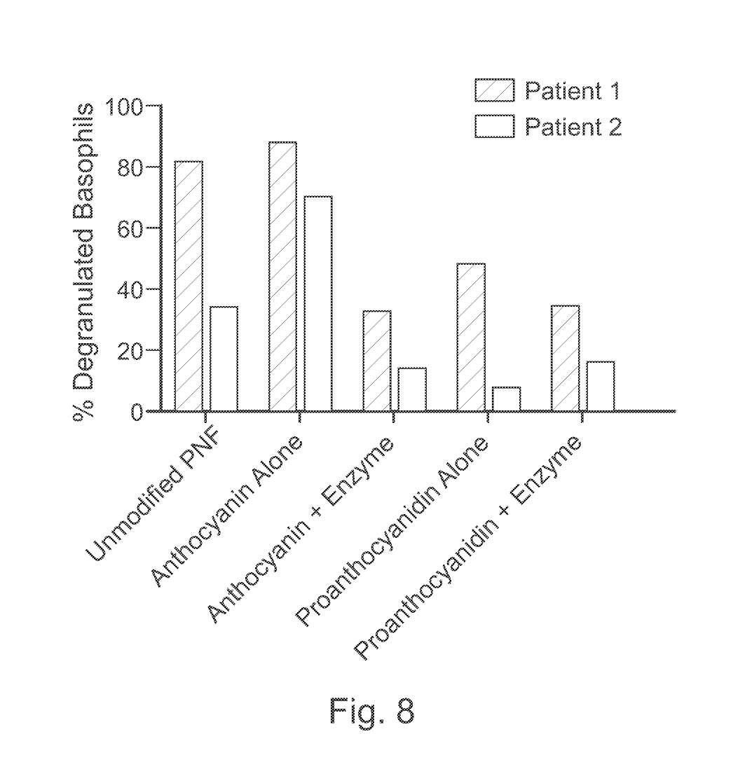

Data from blood draws of two additional peanut allergenic patients is summarized in FIG. 8.

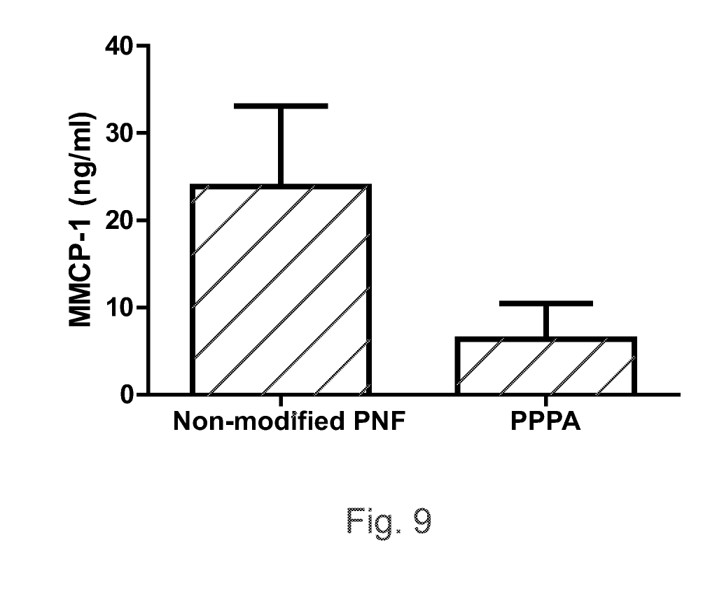

The basophil assay was successfully adapted to allow testing of the modified peanut flours in the solid phase, since the peanut proteins in the modified flours are largely insoluble. Modified peanut flours developed using proanthocyanidin-rich cranberry juice concentrate [at two dilutions, note that 2× is more concentrated than 5×] showed less degranulation, compared to treatments provoked with unmodified peanut flour preparations or peanut flours complexed with black currant polyphenolics after sampling blood from seven peanut allergic patients (FIG. 7). (The highly concentrated, viscous black currant juice concentrate was diluted to comparable total polyphenolic content as 5× cranberry juice concentrate 1.55 and 1.4 mg/mL, respectively). Preliminary studies in small groups (n=4 per group) of mice indicate peanut protein-phytoactive aggregates (PPPA) cause less mast cell degranulation than non-modified peanut flour during an oral challenge in allergic mice. Briefly, C3H/HeJ mice were made allergic to peanut proteins then challenged with 50 mg of PPPA (i.e. cranberry-PNF) or peanut flour (PNF). Mice were bled 45 minutes after challenge and serum was assayed for mouse mast cell protease-1 (MMCP-1) as a marker of degranulation. PPPA-challenged mice had much lower levels of MMCP-1 in their sera than mice challenged with non-modified PN (FIG. 9).

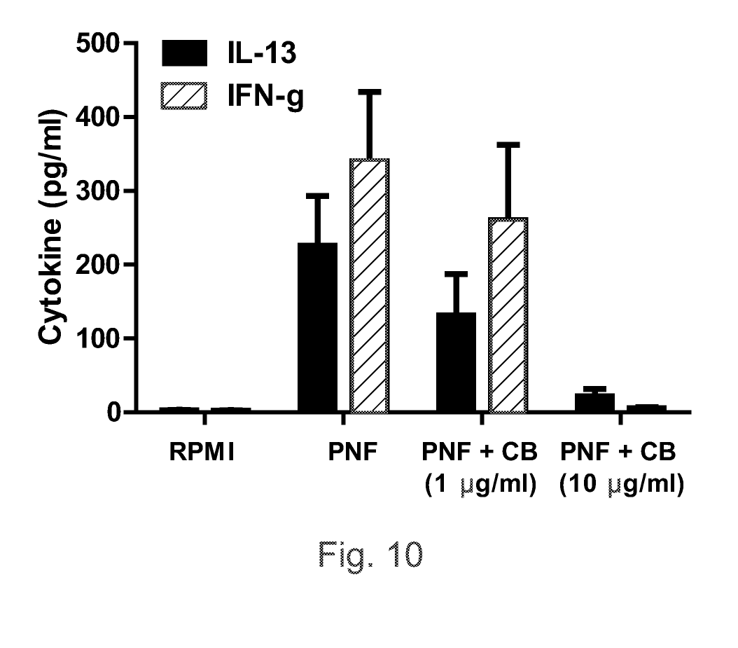

Additional preliminary studies using spleen cells from C3H/HeJ mice were conducted to examine effects of cranberry juice polyphenolics on T cell cytokine secretion. Briefly, spleen cells from peanut-allergic mice were cultured in the presence of: no stimulant (RPMI media alone); 200 μg/ml peanut proteins (PNF); 200 μg/ml PN complexed with cranberry juice (CB) at 1 μg/ml; 200 μg/ml PN complexed with CB at 10 μg/ml. After 96 hours, culture supernatants were collected and analyzed by ELISA for IL-13 and IFN-gamma, representing Th2 and Th1-type cytokines. The results indicate that there is a dose-dependent suppression of both L-13 and IFN-gamma attributable to CB (FIG. 10).

Stronger, covalently bound cranberry fruit derived polyphenolics to peanut epitopes may be engineered by creating the polyphenol-protein complexes in the presence of a natural tyrosinase enzyme.

Thus, an efficient, cost-effective technology for modifying milled peanut flour by complexing with fruit polyphenols, in particular, oligomeric proanthocyanidins, results in creation of a polyphenol-fortified peanut protein edible matrix with potential utility in immunotherapy and novel functional food applications. Reduced allergenicity indicated in these in vitro assays is to be tested in mice to provide in vivo evidence of their hypoallergenicity and potential utility as OIT agents.

The effects of dietary polyphenols on allergic reactions have been reported. To date, these studies have almost exclusively focused on polyphenolic extracts which are often purified, smaller molecular weight chemicals (quercetin, catechin monomers, luteolin). Such extracts alone have shown great potential in preventing and ameliorating allergic reactions including food allergies. Singh, A., S. Holvoet and A. Mercenier. 2011. Dietary polyphenols in the prevention and treatment of allergic diseases. Clinical & Experimental Allergy 41: 1346-1359. Related to this research, a standardized traditional Chinese herbal medicine has been shown to desensitize peanut allergic mice, and many of the active molecules in this herbal mixture consist of polyphenolics interacting synergistically. Nowak-Wegrzyn. A. and H. Sampson. 2011. Future therapies for food allergies. J. Allergy Clin Immunol 127:558-573.

The polyphenolic-fortified peanut flours described herein may mediate peanut allergy by masking epitopes and/or reacting with the immune system in similar ways. The technology combines the proven benefits of oral immunotherapy using peanut flour and the benefits of polyphenolic extracts, to create a unique material, which concentrates and complexes the protective and bioactive polyphenolics, binding them fast to the peanut flour epitopes prior to administration. In addition to masking epitopes, the polyphenolics could act synergistically to mitigate the allergic response. Furthermore, unlike purified or recombinant allergenic proteins, the treated solid protein rich food stuffs of the present invention are likely to better mimic the native food allergen because they retain much of the natural structure and matrix of the allergens.

This system will permit large scale, economically-feasible modification of peanut flour which can be easily adopted for both the food industry and for clinical applications. (Use of the polyphenolic-fortified peanut flour for OIT in humans will, however, first require an Investigational New Drug (IND) application with the FDA). The natural phytochemical mixtures employed in the process, including monomers and higher molecular weight oligomers and polymers from fruits, are capable of thoroughly enveloping protein epitopes and modifying allergenicity in a much more robust manner than any previous reports with purified chemical isolates.

While current allergenicity tests to date have exemplified on peanuts, polyphenolics have been successfully complexed with other protein-rich matrices such as whey, soy, wheat bran, pea, sweet potato, and hemp. Thus, the technology has full potential to alleviate other kinds of food allergies (e.g. milk, soy) using the same strategies, and will deliver cost effective and versatile production advantages. In addition to the functional food market, the technology has strong application in the clinical arena for physicians seeking potentially safer OIT strategies. Because the process uses all GRAS ingredients; no solvents or sophisticated laboratory instrumentation, can be produced in minimal facilities, and creates a reproducible, shelf-stable delivery system, the technology may be readily adopted for tailored applications by industry.

The immunotherapy potential of this technology can resonate with the medical profession, thus the technology can be marketed for clinical applications. Other technologies, some extreme, have been attempted to modify allergic proteins for patient desensitization (including genetic modification of food crops with modified protein profiles), but each carries its own risks and policy issues. This technology offers a safe recognized platform for delivery of modified/masked proteins using all GRAS ingredients, no solvents or need for sophisticated instrumentation, and in an easy to use cost effective format. Once the planned in vivo experiments have confirmed attenuated allergenicity, the materials are submitted for skin prick testing and vaccine formulation for allergy immunotherapy. The materials may also be used for sublingual immunotherapy.

The compositions and methods herein are applicable for preparation of protein-enriched products (power bars, nutritional supplements). Moreover, this is a means to provide protein-rich food with the additional advantage of being antioxidant-enriched. This is certainly true for peanut ingredient producers, but as noted previously, the novel process we have for masking food proteins with natural phytochemical constituents can be extended to other food proteins (soy, whey, etc). Further, the natural phytochemicals used to mask the epitopes (e.g. natural fruit polyphenolics) are already recognized in the public eye as safe and health-beneficial components that can enhance the functional value of a product.

A series of polyphenolic-fortified peanut flour complexes with varying dilutions of fruit juice concentrates with and without the presence of tyrosinase enzyme are prepared, in order to deliver formulations with varying degrees of binding (both non-covalent and covalent). These formulations differ in the degree to which the natural polyphenolics will bind to the peanut epitopes, and maybe engineered for gradual release (or not) during digestion.

The protein distribution and IgE binding of phytochemical conjugates are determined using SDS-PAGE and Western Blotting.

The solid phase basophil assay to quantitate the capacity for polyphenol-complexed peanut flours to inhibit degranulation reactions may be further developed, and the optimum polyphenolic source for masking epitopes is identified.

In vivo tests to further validate the efficacy of the orally administered modified polyphenolic-enhanced peanut flour to reduce allergenicity are conducted and its potential as an immunotherapy agent is assessed.

Hypoallergenicity of the modified flours is demonstrated in a mouse model of peanut allergenicity. Mice are sensitized and reactive to peanuts (using an established protocol), then groups are challenged orally (by gavage) with various preparations of modified and unmodified peanut flours. Mice are monitored for allergic symptoms and release of mast cell/basophil mediators into serum (i.e. histamine, mast cell protease, leukotrienes, etc.).

The efficacy of immunotherapy using polyphenolic-fortified flours is determined in peanut-allergic mice. Mice made allergic to peanut are given modified peanut flours by oral gavage for 4 weeks. A peanut challenge is performed to assess if the mice are desensitized and no longer have allergic reactions to peanuts. Immunologic changes at the T cell level (cytokine production, regulatory T cells, etc) and B cell level (IgE, IgG, IgA) are examined also.

It is to be understood that, while the invention has been described in conjunction with the detailed description, thereof, the foregoing description is intended to illustrate and not limit the scope of the invention. Other aspects, advantages, and modifications of the invention are within the scope of the claims set forth below. All publications, patents, and patent applications cited in this specification are herein incorporated by reference as if each individual publication or patent application were specifically and individually indicated to be incorporated by reference.

1. A solid foodstuff with reduced allergenicity which comprises a solid protein rich ingredient treated with fruit/vegetable polyphenolic phytochemicals.

2. The solid foodstuff of claim 1, wherein the solid protein rich ingredient is a nut or legume protein.

3. The solid foodstuff of claim 2, wherein the legume protein is a peanut protein.

4. The solid foodstuff of claim 3, wherein the peanut protein is a peanut flour.

5-13. (canceled)

14. The solid foodstuff of claim 1, wherein the fruit/vegetable polyphenolic phytochemicals are from a berry extract.

15. The solid foodstuff of claim 14, wherein the berry extract is a cranberry extract.

16. The solid foodstuff of claim 14, wherein the berry extract is a black currant extract.

17. An infant formula prepared with the solid foodstuff of claim 1.

18. A method for reducing the allergenicity of a solid protein rich foodstuff which comprises treating the solid protein rich foodstuff with fruit/vegetable polyphenolic phytochemicals.

19. The method of claim 18, wherein the solid protein rich foodstuff is treated by mixing with a juice or extract rich in fruit/vegetable polyphenolic phytochemicals to form a complex between the solid protein rich foodstuff and the fruit/vegetable polyphenolic phytochemicals.

20. The method of claim 19, which further comprises centrifugation and lyophilization of the complex between the solid protein rich foodstuff and the fruit/vegetable polyphenolic phytochemicals.

21. The method of claim 19, which further comprises centrifugation, and drying or freeze drying (lyophilization) of the complex between the solid protein rich foodstuff and the fruit/vegetable polyphenolic phytochemicals.

22. The method of claim 18, wherein the solid protein rich ingredient is a nut or legume protein.

23-33. (canceled)

34. The method of claim 18, wherein the fruit/vegetable polyphenolic phytochemicals are from a berry extract.

35. The method of claim 34, wherein the berry extract is a cranberry extract.

36. The method of claim 34, wherein the berry extract is a black currant extract.

37. The method of claim 18, wherein the solid protein rich ingredient is a soy protein or a milk protein and the method is used to prepare an infant formula.

38. A method of preparing an infant formula or baby food with reduced allergenicity which comprises treating a solid protein rich foodstuff with fruit/vegetable polyphenolic phytochemicals so as to form a complex between the solid protein rich foodstuff and the fruit/vegetable polyphenolic phytochemicals; and using the complex to prepare the infant formula or baby food with reduced allergenicity.

39. A method of reducing an allergic reaction to a solid protein rich foodstuff in a subject which comprises
(a) treating the solid protein rich foodstuff with fruit/vegetable polyphenolic phytochemicals to form a complex between the solid protein rich foodstuff and the fruit/vegetable polyphenolic phytochemicals;
(b) administering the complex to the subject so as to build a tolerance to the protein rich foodstuff thereby reducing the allergic reaction to the protein rich foodstuff.

40. The method of claim 39, wherein the subject is an infant and the solid protein rich food stuff is a milk protein or a soy protein.
